(19) United States
(12) Patent Application Publication
(10) Pub. No.: US 2001/0042322 A1
(43) Pub. Date: Nov. 22, 2001

(54) Insole

(76) Inventor: Frank Friedrich Grohninger, Oberbexbach (DE)

Correspondence Address: HENRY M FEIEREISEN, 350 FIFTH AVENUE, SUITE 3220, NEW YORK, NY, 10118

(21) Appl. No.: 09/887,397

(22) Filed: Jun. 22, 2001

Related U.S. Application Data
(63) Continuation of application No. PCT/DE99/03403, filed on Oct. 25, 1999.

(30) Foreign Application Priority Data
Dec. 23, 1998 (DE) 298 22 941.2

(51) Int. Cl. A43B 7/14; A61F 5/14; A43B 13/20; A43B 13/38

(52) U.S. Cl. 36/43; 36/72 R; 36/71; 36/29; 36/153

(57) ABSTRACT

An insole which extends from the toes to the heel includes at least one fluid-filled container; a control unit for enabling a user to increase or reduce an amount of fluid in the container; and a stirrup-shaped device for immobilizing the talocalcaneonavicular joint and for controlling and/or stabilizing the ankle joint. The insole is a prefabricated insole selectable from a range of differently shaped insoles for the various forms of foot prophylaxis and the various shoe sizes. This enables users to adapt the prefabricated insoles suitable for their shoe size and form of foot prophylaxis to their own personal needs.

### CROSS-REFERENCES TO RELATED APPLICATIONS

This application is a continuation of prior filed copending PCT International application no. PCT/DE99/03403, filed Oct. 25, 1999.

This application claims the priority of German Patent Application Ser. No. 298 22 941.2, filed Dec. 23, 1998, the subject matter of which is incorporated herein by reference.

### BACKGROUND OF THE INVENTION

The present invention relates, in general, to an insole for shoes, and more particularly to a shoe insole which extends from the toes to the heel and includes at least one fluid-filled container.

European Pat. No. EP 0 060 353 A 1 describes an insole for use especially by people who have to stand for long periods. The fluid-filled containers serve to transmit load stresses from the foot's support points to large-surface parts of the insole, and simultaneously massage those areas of the sole in contact with them. However, these insoles cannot be adapted to the user's individual needs.

European Pat. No. EP 0 434 076 A2 describes an insole which has transverse profiling running transversely to the longitudinal direction of the shoe, and includes one or more damping elements in the heel region and/or in the forefoot region.

German Pat. No. DE 298 05 396 U1 describes a shoe with an exchangeable insole, with the upper part of the shoe being higher than normal for the size of the shoe so as to make the shoe suitable for the insertion of an exchangeable, relatively thick anatomically shaped insole.

German Pat. No. DE 195 40 567 C1 discloses a shoe with a removable footbed to allow, when necessary, to combine the shoe's footbed with a special orthopedic half insole.

A modular system for an insole is known from German Pat. No. DE 298 10 518 U1, which permits a high degree of flexibility in the manufacturing of customized insoles. However, it is highly unlikely that a layman will be able to take the necessary measurements and assemble the product.

German Pat. No. DE 90 00 041 U1 describes an insole with adjustable and exchangeable support height. The insole, which is known per se, is of firm material combined with an elastic upper layer in which there are hollow, non-connected ribs for insertion of the support element.

U.S. Pat. No. 4,665,576 A describes a ski boot provided with several exchangeable insoles that are beveled in various ways.

International applications WO/9500047 and WO/9856272 disclose insoles which have a container and with which the degree of plantar arch support can be adjusted by varying the pressure in the container.

German Pat. No. DE 24 60 034 A 1 describes a sports shoe with a gas or air cushion sac integrated in the sole. In like manner, U.S. Pat. No. 4,008,530 A describes a shoe with an air cavity integrated in the sole.

For prophylactic purposes and in cases of certain orthopedic indications, it is also known to make footprints, which are used to make customized insoles. These insoles can then be inserted into shoes. However, customizing insoles in this way is relatively tedious and very expensive. With increasing life expectation and an increasing number of minor, wear-induced foot disorders, the importance of foot prophylaxis is becoming ever greater. This applies in particular in cases where stress loads are high, e.g. due to sport. However, such prophylaxis would be unaffordable with conventional procedures.

It would therefore be desirable and advantageous to provide an improved low-cost insole which obviates prior art shortcomings and which is suitable for foot prophylaxis and adaptable to the needs of the user.

### SUMMARY OF THE INVENTION

According to one aspect of the present invention, an insole extending from toes to heels, includes at least one fluid-filled container; a control unit for enabling a user to increase or reduce an amount of fluid in the container; and a stirrup-shaped device for immobilizing the talocalcaneonavicular joint and for controlling and/or stabilizing the ankle joint.

These measures significantly reduce the risk of supination and pronation, and are thus especially beneficial in the case of sports such as soccer and cross-country running, and also for rehabilitation shoes when the patient has suffered a supination trauma and distortions in the ankle joint area. Immobilizing the talocalcaneonavicular joint in this way is also recommended for sports which heavily stress this joint and the ankle joint, e.g. soccer, American football, rugby, handball, basketball, tennis, etc.

The container can extend over the entire insole, or only over part of it. In the former case, the insole itself can be designed as a container. Since the user is able to vary the hardness of the insole in the area of the container, he/she can adapt the insoles to suit his/her personal needs.

According to another feature of the present invention, the control unit may include a valve. Suitably, the valve is located at a side of the insole.

According to another feature of the present invention, the container may be made of a gel or a foamed plastic. Suitably, the container may be made with a graduation for indication of the amount of fluid in the container. The graduation may include graduation marks running transversely to the longitudinal axis of the insole. The container may suitably disposed in the heel region and/or in the area of the lateral longitudinal arch and/or in the area extending from the fifth metatarsal head to the first metatarsal head.

According to another feature of the present invention, the insole is a prefabricated insole selectable from a range of insoles for different forms of foot prophylaxis and different shoe sizes. Customized insoles are thus only required in case of medical indications, while simple foot prophylaxis and protective therapy for athletes' feet can be undertaken cost efficiently with a modular system of prefabricated insoles designed for specific forms of prophylaxis. This means that for each shoe size, there is a range of insoles available for common types of foot prophylaxis. While in a shoe store, the user can snugly fit insoles into the shoe, test them, and choose the ones that best suit the needs of his/her feet. The user can then adjust the hardness to his/her own needs by adding or withdrawing fluid.

Suitably, the insoles may be used for standard foot prophylaxis or for various fallen-arch processes.

It is also conceivable to provide a metatarsal truss pad.

According to another feature of the present invention, a shin-bone protector may be integrated in the stirrup-shaped device. It is furthermore useful that the heel region of the insole is raised by an insertion wedge and/or that the area between forefoot and distal tarsus can be designed flatter. This provides relief for the Achilles tendon.

It is also conceivable to provide insoles with different degrees of hardness and/or insoles with zones that differ in hardness.

### BRIEF DESCRIPTION OF THE DRAWING

Other features and advantages of the present invention will be more readily apparent upon reading the following description of preferred exemplified embodiments of the invention with reference to the accompanying drawing, in which:

FIG. 1a shows a top view of an insole according to the invention;

FIG. 1b is a sectional view of the insole, taken along the line I-I of FIG. 1a.

FIG. 2a is a top and front perspective illustration of a variation of an insole according to the present invention; and

FIG. 2b is a rear view of the insole of FIG. 2a.

### DETAILED DESCRIPTION OF PREFERRED EMBODIMENTS

Throughout all the Figures, same or corresponding elements are generally indicated by same reference numerals.

Insoles are provided for all common shoe sizes and configured for snug fit in the shoe. The insoles accommodate the sole of the foot in a form fit, and intended for cushioning and supporting the foot structures that are exposed to stress. The invention provides for a range of insoles as listed below (using shoe-size 40 as an example):

40 A: Modular insole for standard foot prophylaxis; Modular insole for diverse degenerative symptoms in the foot structures, the shape of the insole varying to suit the specific symptom;

40B: Fallen plantar arch (early stage) as a result of the old age;

40C: Fallen plantar arch (medium stage) as a result of old age;

40D: Fallen plantar arch (advanced stage) as a result of old age;

40E: Modular insole with stirrup-type stabilization and control of the ankle joint (also in combination with shin-bone protector);

40F: Modular insole for standard foot prophylaxis with heel raising.

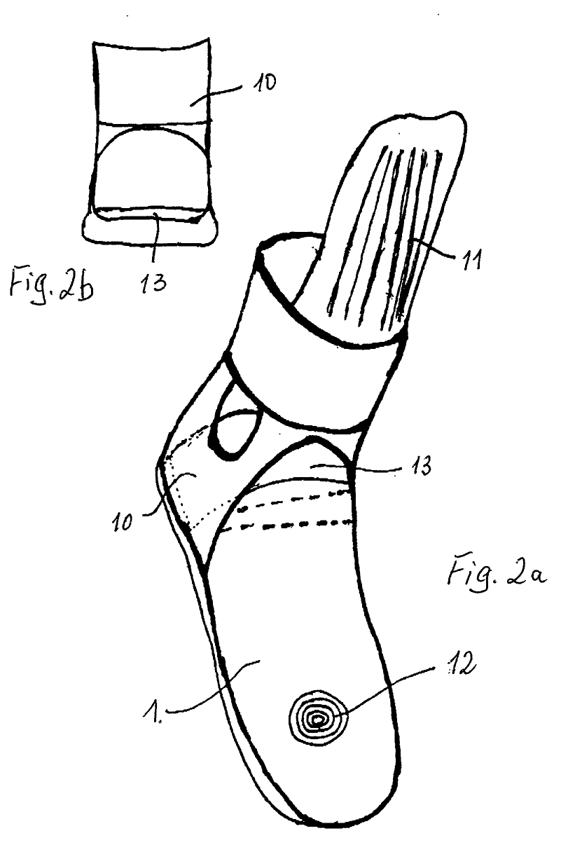

All insole types are provided in different degrees of hardness and with zones that differ in hardness, and all may include a metatarsal truss pad, such as a metatarsal truss pad 12, shown, by way of example, in FIG. 2a.

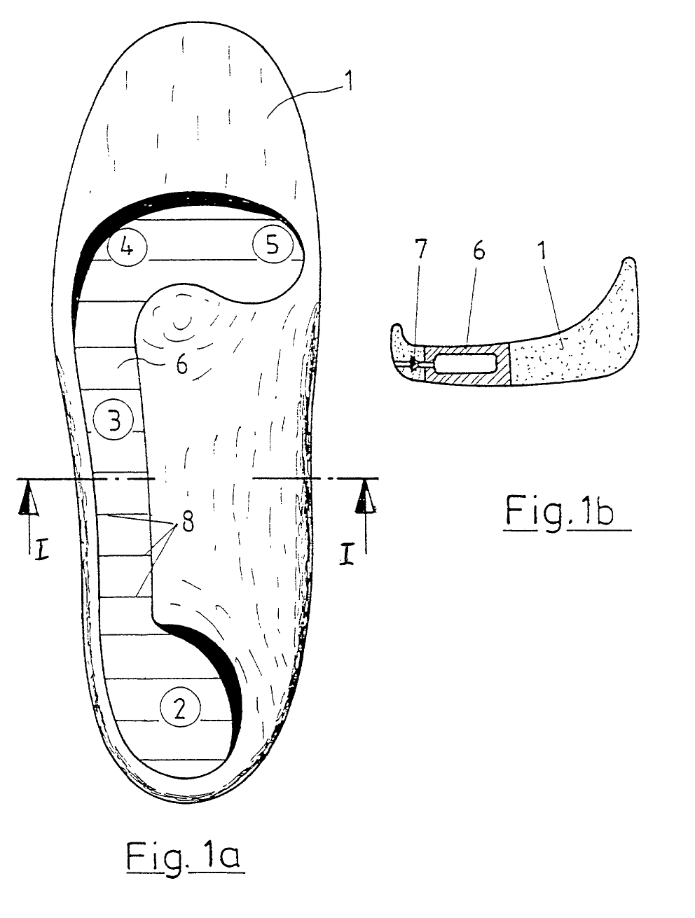

Turning now to the drawing, and in particular to FIG. 1, there is shown a top view of an insole 1 according to the invention. The insole 1 has a container 6 extending from a heel region 2 through a region of the lateral longitudinal arch 3 to a region of the fifth metatarsal head 4 and from there to a region of the first metatarsal head 5. The container 6 is filled with a gaseous (e.g. air) or liquid fluid, preferably a water-based liquid, and is preferably made of a soft material which is impermeable to liquids and gases, adapts to the shape of the foot and has comfortable support properties. Examples as material for the container 6 include, e.g., a gel or a foamed plastic, such as foamed ethylene. Of course, a person skilled in the art is aware of a wide range of gels or foamed plastics and will utilize the type of material which is best adapted to the particular application in which the invention is to be used.

As shown in FIG. 1b, which is a sectional view of the insole, taken along the line I-I of FIG. 1, a valve 7 is provided at a side of the insole 1 for controlling, i.e. increasing or reducing, the amount of fluid in the container 6. This can be done, for example, by means of a syringe, which is insertable into the valve 7, for injection of fluid into the container 6 or withdrawal of fluid from the container 6.

To enable a user to readjust the amount of fluid in the container 6, it is useful to provide a graduation 8, which comprises, for example, graduation marks running transversely to the longitudinal direction of the insole 1. In this way, the user is able to select a suitable insole for a shoe size and for foot prophylaxis, and then to adapt it individually to own personal needs.

As an alternative, the container 6 may also be located only in the heel region or in the region of the lateral longitudinal arch, or from the region of the fifth metatarsal head to the first metatarsal head, or in any combination of these regions.

Turning now to FIG. 2a, there is shown a top and front perspective illustration of a variation of the insole 1 according to the present invention. Parts corresponding with those in FIG. 1a are denoted by identical reference numerals and not explained again. In this embodiment, provision is made for a stirrup-shaped device 10 for immobilizing the talocalcaneonavicular joint and for controlling andlor stabilizing the ankle joint of a human being. The stirrup-shaped device 10 is thus secured to a rear portion of the insole 1 and projects upwards. Suitably, a shin-bone protector 11 is integrated in the stirrup-shaped device 10. In a forward area thereof, the insole 1 includes a metatarsal truss pad 12 for providing an elevation in the metatarsal area whereas in the heel region, the insole 1 is raised by an insertion wedge 13 which is flattened towards the forward part. As an alternative, the area between forefoot and distal tarsus may also be designed flatter. FIG. 2b shows a rear view of the insole 1. Of course, the provision of the shin-bone protector 11, metatarsal truss pad 12 and insertion wedge 13 are optional and any combination should be considered within the scope of the present invention.

While the invention has been illustrated and described as embodied in an insole, it is not intended to be limited to the details shown since various modifications and structural changes may be made without departing in any way from the spirit of the present invention.

What is claimed as new and desired to be protected by Letters Patent is set forth in the appended claims:

What is claimed is:

1. An insole extending from toes to heels, comprising:
at least one fluid-filled container;
control means for enabling a user to increase or reduce an amount of fluid in the container; and
a stirrup-shaped device for immobilizing a talocalcaneonavicular joint and for controlling and/or stabilizing an ankle joint.

2. The insole of claim 1, wherein the control means includes a valve.

3. The insole of claim 2, wherein the valve is located at a side of the insole.

4. The insole of the claim 1, wherein the container is made of a material selected from the group consisting of gel and foamed plastic.

5. The insole of claim 1, wherein the container has a graduation for indication of the amount of fluid in the container.

6. The insole of claim 5, wherein the graduation has graduation marks running transversely to a longitudinal axis of the insole.

7. The insole of claim 1, wherein the container is located at least in a region selected from the group consisting of heel, lateral longitudinal arch, and an area extending from fifth metatarsal head to first metatarsal head.

8. The insole of claim 1, being prefabricated and selected from a range of insoles for different forms of foot prophylaxis and different shoe sizes.

9. The insole of claim 8, for use for one of standard foot prophylaxis and various degenerative fallen-arch processes.

10. The insole of claim 1, and further comprising a metatarsal truss pad.

11. The insole of claim 1, and further comprising a shin-bone protector integrated in the stirrup-shaped device.

12. The insole of claim 1, and further comprising an insertion wedge for elevating a heel region of the insole.

13. The insole of claim 1, wherein an area between forefoot and distal tarsus is flattened for elevating a heel region of the insole.

14. The insole of claim 1, wherein the insole has zones differing in hardness.

15. A kit, comprising a plurality of insoles, each of the insoles having at least one fluid-filled container; control means for enabling a user to increase or reduce an amount of fluid in the container; and a stirrup-shaped device for immobilizing a talocalcaneonavicular joint and for controlling and/or stabilizing an ankle joint, wherein the insoles have different hardness.
